(19) United States
(12) Patent Application Publication
(10) Pub. No.: US 2022/0259555 A1
(43) Pub. Date: Aug. 18, 2022

(54) Separation of Immune Cells by Multiple Microfluidic Devices

(71) Applicant: Applied Cells Inc., Santa Clara, CA (US)

(72) Inventors: Liping Yu, San Jose, CA (US); Silin Sa, San Jose, CA (US)

(21) Appl. No.: 17/672,031

(22) Filed: Feb. 15, 2022

Related U.S. Application Data
(60) Provisional application No. 63/149,612, filed on Feb. 15, 2021.

(51) Int. Cl. C12N 5/00; C12N 13/00; B01L 3/00

(52) U.S. Cl. CPC C12N 5/0093; C12N 13/00; B01L 3/502761; B01L 2400/0439; B01L 2300/042; B01L 2300/0864; B01L 2300/14; B01L 2200/0652

(57) ABSTRACT

A method for extracting or enriching immune cells in a fluid sample, which contains immune and cancer cells and debris, includes the steps introducing the fluid sample into a first microfluidic device as two streams along two sidewalls thereof; applying a first power to the first microfluidic device to exert a first acoustic radiation pressure to produce a first output fluid having a higher relative fraction of the cancer cells than the fluid sample and a second output fluid having a lower relative fraction of the cancer cells than the fluid sample; introducing the second output fluid into a second microfluidic device as two streams along two sidewalls thereof; and applying a second power, which is higher than the first power, to the second microfluidic device to exert a second acoustic radiation pressure to produce a third output fluid having a higher relative fraction of the immune cells than the fluid sample.

### BACKGROUND

The present invention relates to a method for separating particles in a fluid, and more particularly, to an acoustic separation method for separating particles or biological entities by two or more microfluidic devices.

Acoustic particle separation method for extracting or separating various biological cells from a fluid sample, such as blood, is of great interest in biological and biomedical applications. The method uses acoustic radiation pressure to move small and large particles suspended in a fluid through a microfluidic device to separate them by size. While the acoustic particle separation method provides a convenient, label-free approach for separating biological cells, the method is mostly limited to separating particles or cells into two groups because of the difficulties in manipulating particles of assorted sizes in a consistent manner.

Guldiken et al. (Sheathless size-based acoustic particle separation, Sensors 12, 905-922 (2012)) disclose a microfluidic device that can separate small, medium, and large particles by using a two-node acoustic standing wave as shown in FIG. 1A. Such approach may be limited to a narrow range of the size ratio of small/medium/large particles and is unlikely to produce end samples with high purities expected from the typical acoustic separation method.

Adams et al. (Tunable acoustophoretic band-pass particle sorter, Applied Physics Letters 97, 064103 (2010)) disclose another microfluidic device that can separate small, medium, and large particles by a two-stage separating process as shown in FIG. 1B. Since the sample flow rates in the first and second stages are coupled, the overall maximum sample flow rate through the two-stage microfluidic device is limited by one of the two stages, thereby limiting the device efficiency.

For the foregoing reason, there is a need for an acoustic separation method that can reliably separate particles or cells with high efficiency.

### SUMMARY

The present invention is directed to a method for separating biological entities in a fluid comprising the steps of introducing an initial fluid sample into a first microfluidic device as two streams along two sidewalls of a first linear channel at an upstream end thereof with a first buffer fluid interposed between the two streams of the initial fluid sample, which includes tumor cells and tumor infiltrating lymphocyte (TIL) cells; applying a first power to the first microfluidic device to exert a first acoustic radiation pressure on the initial fluid sample and the first buffer fluid flowing in the first linear channel to produce a first output fluid sample exiting the first microfluidic device along a center of the first linear channel and a second output fluid sample exiting the first microfluidic device along the two sidewalls of the first linear channel, the first output fluid sample having a higher relative fraction of the tumor cells than the initial fluid sample and the second output fluid sample having a lower relative fraction of the tumor cells than the initial fluid sample; flowing the second output fluid sample from the first microfluidic device into a flow connector to accumulate the second output fluid sample; flowing the second output fluid sample accumulated in the flow connector into a second microfluidic device as two streams along two sidewalls of a second linear channel at an upstream end thereof with a second buffer fluid interposed between the two streams of the second output fluid sample; and applying a second power to the second microfluidic device to exert a second acoustic radiation pressure on the second output fluid sample and the second buffer fluid flowing in the second linear channel to produce a third output fluid sample exiting the second microfluidic device along a center of the second linear channel, the third output fluid having a higher relative fraction of TIL cells than the initial fluid sample. The second power is higher than the first power. A flow rate of the second output fluid sample exiting the first microfluidic device is decoupled from a flow rate of the second output fluid sample entering the second microfluidic device.

According to another aspect of the present invention, a method for separating biological entities in a fluid comprises the steps of introducing an initial fluid sample into a first microfluidic device as two streams along two sidewalls of a first linear channel at an upstream end thereof with a first buffer fluid interposed between the two streams of the initial fluid sample, which includes cancer cells and peripheral blood mononuclear cells (PBMCs); applying a first power to the first microfluidic device to exert a first acoustic radiation pressure on the initial fluid sample and the first buffer fluid flowing in the first linear channel to produce a first output fluid sample exiting the first microfluidic device along a center of the first linear channel and a second output fluid sample exiting the first microfluidic device along the two sidewalls of the first linear channel, the first output fluid sample having a higher relative fraction of the cancer cells than the initial fluid sample and the second output fluid sample having a lower relative fraction of the cancer cells than the initial fluid sample; flowing the second output fluid sample from the first microfluidic device into a flow connector to accumulate the second output fluid sample; flowing the second output fluid sample accumulated in the flow connector into a second microfluidic device as two streams along two sidewalls of a second linear channel at an upstream end thereof with a second buffer fluid interposed between the two streams of the second output fluid sample; and applying a second power to the second microfluidic device to exert a second acoustic radiation pressure on the second output fluid sample and the second buffer fluid flowing in the second linear channel to produce a third output fluid sample exiting the second microfluidic device along a center of the second linear channel, the third output fluid having a higher relative fraction of PBMCs than the initial fluid sample. The second power is higher than the first power. A flow rate of the second output fluid sample exiting the first microfluidic device is decoupled from a flow rate of the second output fluid sample entering the second microfluidic device.

### BRIEF DESCRIPTION OF THE DRAWINGS

These and other features, aspects, and advantages of the present invention will become better understood with regard to the following description, appended claims, and accompanying drawings where:

FIG. 1A shows a prior art microfluidic device using acoustic radiation pressure for separating small, medium, and large particles in a fluid sample;

FIG. 1B shows another prior art microfluidic device using a two-stage process for separating small, medium, and large particles in a fluid sample;

FIG. 2A is a top view of a microfluidic device for separating biological entities in accordance with an embodiment of the present invention;

FIGS. 2B and 2C are cross-sectional views of the microfluidic device showing alternative positions of the inlet ports;

FIGS. 2D and 2E are cross-sectional views of the microfluidic device at the upstream end of the fluidic channel illustrating alternative positions for attachment of piezoelectric transducers;

FIGS. 2F and 2G are cross-sectional views of the microfluidic device at the downstream end of the fluidic channel illustrating alternative positions for attachment of piezoelectric transducers;

FIG. 3 illustrates operation of the microfluidic device under single pressure node condition;

FIG. 4 illustrates two microfluidic devices connected in a serial configuration for acoustic separation;

FIG. 5 illustrates three microfluidic devices connected in a cascading configuration for acoustic separation;

FIG. 6 illustrates two microfluidic devices connected in another serial configuration for acoustic separation;

FIG. 7 illustrates a first type of flow connector for decoupling the flow rates of two microfluidic devices connected in series;

FIG. 8 illustrates a second type of flow connector for decoupling the flow rates of two microfluidic devices connected in series;

FIG. 9 illustrates a third type of flow connector for decoupling the flow rates of two microfluidic devices connected in series;

FIG. 10 illustrates a process for extracting or enriching tumor infiltrating lymphocyte (TIL) cells suspended in a fluid sample; and

FIG. 11 illustrates a process for extracting or enriching peripheral blood mononuclear cells (PBMCs) suspended in a fluid sample.

For purposes of clarity and brevity, like elements and components will bear the same designations and numbering throughout the Figures, which are not necessarily drawn to scale.

### DETAILED DESCRIPTION

In the Summary above and in the Detailed Description, and the claims below, and in the accompanying drawings, reference is made to particular features (including method steps) of the invention. It is to be understood that the disclosure of the invention in this specification includes all possible combinations of such particular features. For example, where a particular feature is disclosed in the context of a particular aspect or embodiment of the invention, or a particular claim, that feature can also be used, to the extent possible, in combination with and/or in the context of other particular aspects and embodiments of the invention, and in the invention generally.

Where reference is made herein to a method comprising two or more defined steps, the defined steps can be carried out in any order or simultaneously, except where the context excludes that possibility, and the method can include one or more other steps which are carried out before any of the defined steps, between two of the defined steps, or after all the defined steps, except where the context excludes that possibility.

The term “at least” followed by a number is used herein to denote the start of a range beginning with that number, which may be a range having an upper limit or no upper limit, depending on the variable being defined. For example, “at least 1” means 1 or more than 1. The term “at most” followed by a number is used herein to denote the end of a range ending with that number, which may be a range having 1 or 0 as its lower limit, or a range having no lower limit, depending upon the variable being defined. For example, “at most 4” means 4 or less than 4, and “at most 40%” means 40% or less than 40%. When, in this specification, a range is given as “a first number to a second number” or “a first number-a second number,” this means a range whose lower limit is the first number and whose upper limit is the second number. For example, “25 to 100 nm” means a range whose lower limit is 25 nm and whose upper limit is 100 nm.

The term “acoustic contrast” may be used herein to mean the relative difference in the density/compressibility ratio between an object and the host medium with regard to the ability to manipulate its position with acoustic radiation pressure. Objects having higher density/compressibility ratios than the host medium may have positive acoustic contrast, which tends to move the objects towards pressure nodes. Conversely, objects having lower density/compressibility ratios than the host medium may have negative acoustic contrast, which tends to move the objects towards pressure antinodes.

The term “biological entities” may be used herein to include cells, bacteria, viruses, molecules, particles including RNA and DNA, cell cluster, bacteria cluster, molecule cluster, and particle cluster.

The term “biological sample” may be used herein to include blood, body fluid, tissue extracted from any part of the body, bone marrow, hair, nail, bone, tooth, liquid and solid from bodily discharge, or surface swab from any part of body. “Entity liquid,” or “fluid sample,” or “sample fluid,” or “liquid sample,” or “sample solution” may include a biological sample in its original liquid form, biological entities being dissolved or dispersed in a buffer liquid, or a biological sample dissociated from its original non-liquid form and dispersed in a buffer fluid. A buffer fluid is a liquid to which biological entities may be dissolved or dispersed without introducing contaminants or unwanted biological entities. Biological entities and biological sample may be obtained from human or animal. Biological entities may also be obtained from plant and environment including air, water and soil. Entity fluid or fluid sample may contain various types of magnetic or optical labels, or one or more chemical reagents that may be added during various steps in accordance with the present invention.

The term “sample flow rate” or “flow rate” may be used herein to represent the volume amount of a fluid sample flowing through a cross section of a channel, or a fluidic part, or a fluidic path, in a unit time.

The term “relative fraction” may be used herein to represent the ratio of a given quantity of biological entities or particles to the quantity of all biological entities or particles present in a fluid sample.

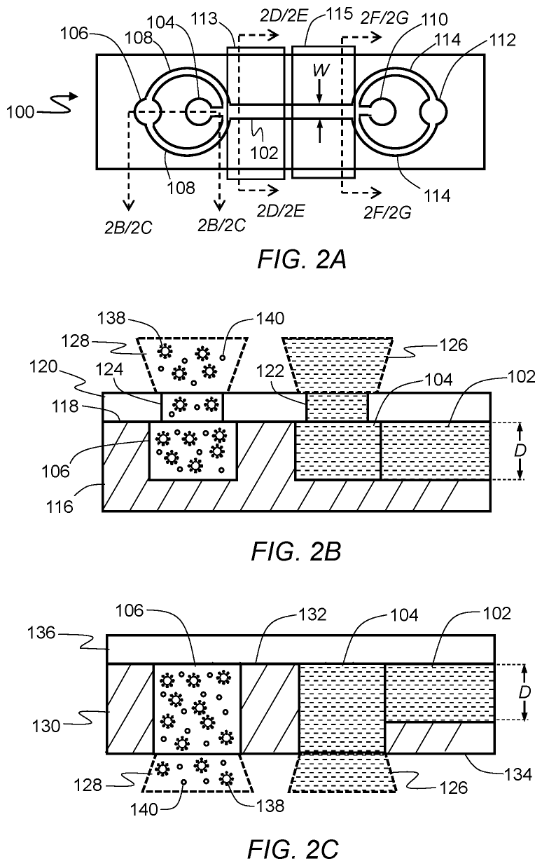

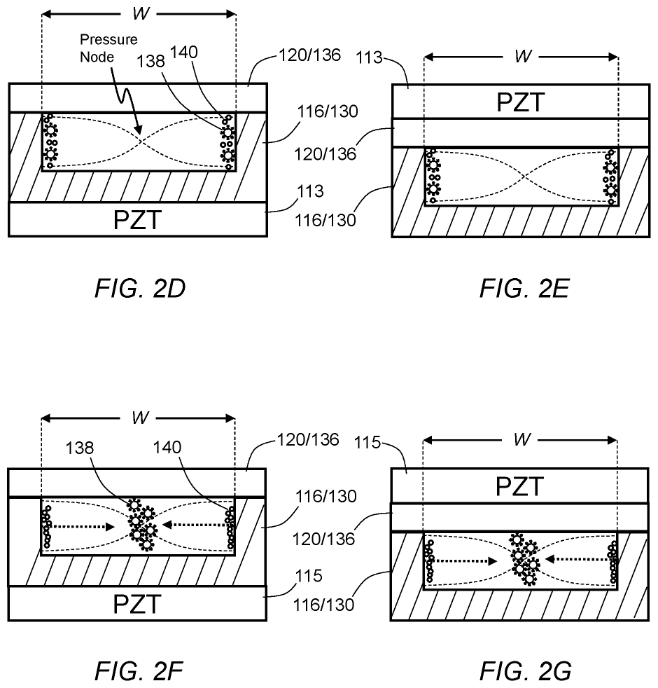

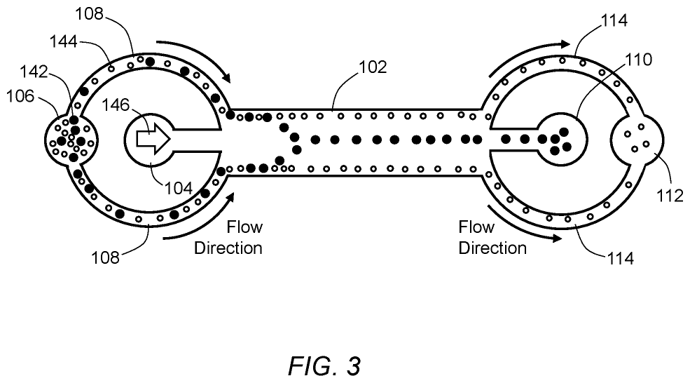

An embodiment of the present invention as applied to a microfluidic device for separating particles or biological entities based on physical size and/or acoustic contrast will now be described with reference to FIGS. 2-3. FIG. 2A is a top view of the microfluidic device 100, which includes a main channel 102, a center inlet port 104 connected to the main channel 102 at the upstream end thereof for introducing a first input fluid into the main channel 102, a side inlet port 106 for introducing a second input fluid into the main channel 102 near the two sidewalls thereof, two side input channels 108 connecting the side inlet port 106 to the main channel 102 at or near the upstream end thereof, a center outlet port 110 connected to the main channel 102 at the downstream end thereof for extracting a first output fluid, a side outlet port 112 for extracting a second output fluid flowing near the two sidewalls of the main channel 102, and two side output channels 114 connecting the side outlet port 112 to the main channel 102 at or near the downstream end thereof. The microfluidic device 100 further includes one or more piezoelectric transducers 113 and 115 for generating the acoustic radiation pressure for acoustic particle separation.

With continuing reference to FIG. 2A, the main channel 102 may have a linear shape with a nominal width, W, between two sidewalls. A portion of the main channel 102 between the center inlet port 104 and the side input channels 108 may be narrower than the nominal width. Likewise, another portion of the main channel 102 between the center outlet port 110 and the side output channels 1114 may be narrower than the nominal width. The width of the side input channels 108 and the width of the side output channels 114 may be narrower than the nominal width of the main channel 102.

The two side input channels 108 connects to the main channel 102 at the two sidewalls thereof, near or at the upstream end. Therefore, the second input fluid, which flows through the two side input channels 108, is introduced into the main channel 102 as two streams flowing near the two sidewalls of the main channel 102. The first input fluid is introduced into the center of the main channel 102 and is squeezed between the two streams of the second input fluid at or near the upstream end of the main channel 102.

The two side output channels 114 connects to the main channel 102 at the two sidewalls thereof, at or near the downstream end. Therefore, the fluid flowing near the two sidewalls at or near the downstream end of the main channel 102 is diverted by the two side output channels 114 to become the second output fluid and exits through the side outlet port 112. The remaining fluid not diverted by the two side output channels 114 becomes the first output fluid and exits through the center outlet port 110.

FIG. 2B is a cross-sectional view of a portion of the microfluidic device 100 showing the center and side inlet ports 104 and 106 in accordance with an embodiment of the present invention. The above-described features 102-114 of the microfluidic device 100 are recessed into a substrate 116 from a top surface 118 thereof. A substrate lid or cover 120 may be attached to the substrate 116 at the top surface 118 thereof and covers the features 102-114 of the microfluidic device 100. The substrate lid or cover 120 includes two holes or openings 122 and 124 aligned to the center and side inlet ports 104 and 106, respectively. The first input fluid 126 and the second input fluid 128 may respectively flow into the center and side inlet ports 104 and 106 through the openings 122 and 124 in the substrate lid or cover 120. The substrate cover 120 may further include two additional holes or openings (not shown) respectively aligned to the center and side outlet ports 110 and 112 for extracting the first and second output fluids. The main channel 102 may have a nominal channel depth, D, measured from the top surface 118.

Alternatively, the center and side inlet ports 104 and 106 may be accessed through the bottom of the microfluidic device 100 as shown in the cross-sectional view of FIG. 2C. Like the embodiment shown in FIG. 2B, the channels 102, 108, 114 and ports 104, 106, 110, 112 of the microfluidic device 100 are recessed into a substrate 130 from a top surface 132 thereof. Additionally, the center and side inlet ports 104 and 106 are further extended to perforate a bottom surface 134 of the substrate 130 for receiving the first and second input fluids 126 and 130. The center and side outlet ports 110 and 112 (not shown in FIG. 2C) may also be further extended to perforate the bottom surface 134 of the substrate 130 for outputting the first and second output fluids. A substrate lid or cover 136 may be attached to the substrate 130 at the top surface 132 thereof and covers the channels 102, 108, 114 and ports 104, 106, 110, 112 of the microfluidic device 100.

While FIGS. 2B and 2C show that the first input fluid 126, which may be a buffer fluid, is introduced into the center inlet port 104 and the second input fluid 128, which may contain particles or biological entities for acoustic separation, is introduced into the side inlet port 106, the first and second input fluids 126 and 128 may alternatively be introduced into the side and center inlet ports 106 and 104, respectively, depending on the operation mode of the microfluidic device 100. Moreover, the second input fluid 128 may contain large particles or biological entities 138 and small particles or biological entities 140 for separation by acoustic radiation pressure. Alternatively, the particles or biological entities 138 and 140 may have sufficiently different acoustic contrasts for acoustic separation.

With continuing reference to FIGS. 2A-2C, the substrates 116/130 may comprise any suitable material, such as but not limited to glass, quartz, fused silica, metal, ceramic material, silicon, silicon carbide, aluminum nitride, titanium carbide, aluminum oxide, zirconium oxide, lithium niobate, magnesium oxide, or any combination thereof. The channels 102, 108, 114 and ports 104, 106, 110, 112 may be formed in the substrate 116/130 by removing material therefrom via any suitable method, such as but not limited to water jet machining, mechanical machining, laser machining, wet etching, plasma etching, or any combination thereof. The substrate cover 120/136 may comprise any suitable material, such as but not limited to glass, quartz, fused silica, metal, polymeric material, ceramic material, silicon, silicon carbide, aluminum nitride, titanium carbide, aluminum oxide, zirconium oxide, lithium niobate, magnesium oxide, or any combination thereof. In an embodiment, the substrate 116/130 and the substrate cover 120/136 are made of the same material. The substrate cover 120/136 may be permanently or irreversibly attached to the substrate 116/130 by any suitable bonding method, such as but not limited to adhesive bonding, fusion bonding, anodic bonding, or any combination thereof

The substrate 116/130 may alternatively comprise a moldable rubber or polymeric material, such as but not limited to polycarbonate or PDMS, that can be molded to form the channels 102, 108, 114 and ports 104, 106, 110, 112 of the microfluidic device 100. When the substrate 116/130 is made of a soft or rubber-like material, such as PDMS or silicone, that lacks structure integrity and may even sag under its own weight, the substrate cover 120/136 made of a relatively stiffer material may be used to support the substrate 116/130.

FIG. 2D shows the cross section of the microfluidic device 100 near the upstream end of the main channel 102 in accordance with an embodiment of the present invention. In the drawing, numerals 113, 116, 120, 130, and 136-140 denote the same components as those shown in FIGS. 2A-2C. Referring now to FIG. 2D, the first piezoelectric transducer 113 in the form of a lead zirconate titanate (PZT) transducer is attached to the exterior or bottom surface of the substrate 116/130. The first piezoelectric transducer 113 may alternatively comprise any suitable piezoelectric material, such as but not limited to potassium niobate, sodium niobate, sodium tungstate, zinc oxide, bismuth ferrite, bismuth titanate, polyvinylidene fluoride, polyvinylidene chloride, polyimide, or any combination thereof. The first piezoelectric transducer 113 may be permanently or irreversibly attached to the bottom surface of the substrate 116/130 by soldering or an adhesive, such as but not limited to epoxy, cyanoacrylate, methacrylate, or any combination thereof.

The first piezoelectric transducer 113 may receive power in the form of an oscillating voltage with a frequency in the range of 100 kHz to 100 MHz to generate acoustic pressure waves in the main channel 102 between two sidewalls when a liquid is present therein. An acoustic standing wave may form in the main channel 102 when the channel width, W, is an integer multiple of one-half wavelength of the acoustic pressure wave, which may depend on the excitation frequency of the power applied to the first piezoelectric transducer 113 and the compressibility and density of the liquid in the main channel 102. FIG. 2D shows that an acoustic standing wave having a half wavelength of W is formed between the two sidewalls of the main channel 102, which results in the formation of single acoustic pressure node at the center of the main channel 102.

The first piezoelectric transducer 113 may alternatively be attached to the exterior or top surface of the substrate cover 120/136 as shown in FIG. 2E by soldering or an adhesive, such as but not limited to epoxy, cyanoacrylate, methacrylate, or any combination thereof.

FIG. 2F shows the cross section of the microfluidic device 100 near the downstream end of the main channel 102 in accordance with an embodiment of the present invention. In the drawing, numerals 115, 116, 120, 130, and 136-140 denote the same components as those shown in FIGS. 2A-2C. Referring now to FIG. 2F, the second piezoelectric transducer 115 in the form of a lead zirconate titanate (PZT) transducer is attached to the exterior or bottom surface of the substrate 116/130. The second piezoelectric transducer 115 may alternatively comprise any suitable piezoelectric material described above for the first piezoelectric transducer 113. The second piezoelectric transducer 115 may be permanently or irreversibly attached to the bottom surface of the substrate 116/130 by soldering or an adhesive as described above.

Like the first piezoelectric transducer 113, the second piezoelectric transducer 115 may receive power in the form of an oscillating voltage with a frequency in the range of 100 kHz to 100 MHz to generate acoustic pressure waves in the main channel 102 between two sidewalls when a liquid is present therein. FIG. 2F shows that an acoustic standing wave having a half wavelength of W is formed between the two sidewalls of the main channel 102, which results in the formation of single acoustic pressure node at the center of the main channel 102.

The second piezoelectric transducer 115 may alternatively be attached to the exterior or top surface of the substrate cover 120/136 as shown in FIG. 2G by soldering or an adhesive, such as but not limited to epoxy, cyanoacrylate, methacrylate, or any combination thereof.

Both of the first and second piezoelectric transducers 113 and 115 may be attached to the bottom surface of the substrate 116/130 or the top surface of the substrate cover 120/136. Alternatively, one of the piezoelectric transducers 113 and 115 may be attached to the bottom surface of the substrate 116/130 while the other one may be attached to the top surface of the substrate cover 120/136.

FIGS. 2D and 2E further show that the particles or biological entities 138 and 140 from the second input fluid 128 flowing along the two sidewalls of the main channel 102 near the upstream end thereof as the second input fluid 128 is being introduced into the main channel 102 via the two side input channels 108. The acoustic radiation pressure may drive the large particles or biological entities 138 towards the pressure node at the center of the main channel 102. By the time the particles or biological entities 138 and 140 reach the downstream end of the main channel 102, as shown in FIGS. IF and 1G, the large particles or biological entities 138 have mostly move to the center of the main channel 102 while the small particles or biological entities 140 mostly remain close to the sidewalls, thereby allowing the small particles or biological entities 140 to be diverted from the main channel 102 via the two side output channels 114.

While FIG. 2A shows the microfluidic device 100 including two piezoelectric transducers 113 and 115, any number of piezoelectric transducers with each covering at least a portion of the main channel 102 may be used. For example, the two piezoelectric transducers 113 and 115 may be merged into a single piezoelectric transducer.

While FIGS. 2D-2G show the formation of single pressure node in the main channel 102, the microfluidic device 100 of the present invention may operate with multiple pressure nodes by adjusting the nominal width of the main channel 102 and/or the excitation frequency of the power applied to the piezoelectric transducer 113 and 115. In an embodiment, all piezoelectric transducers operate at the same frequency. In another embodiment, at least one of the piezoelectric transducers operates at a different frequency from others, resulting in a portion of the main channel 102 having a different number of pressure nodes from other portions. For example and without limitation, the first piezoelectric transducer 113 may operate at a double frequency compared to the second piezoelectric transducer 115, resulting in the upstream and downstream portions of the main channel 102 having two and one pressure node, respectively.

Operation of the microfluidic device 100 under the condition of single pressure node will now be described with reference to FIG. 3. In the drawing, numerals 102-114 denote the same components as those shown in FIG. 2A and the piezoelectric transducers are omitted for reasons of clarity. Referring now to FIG. 3, a fluid sample containing a first type of particles or biological entities 142 and a second type of particles or biological entities 144 is introduced into the side inlet port 106 while a buffer fluid 146 is introduced into the center inlet port 104. The first and second types of particles or biological entities 142 and 144 may have sufficiently different physical sizes and/or acoustic contrasts to allow them to be separated by acoustic radiation pressure. For example, the first type of particles or biological entities 142 may have a larger physical size, and/or a higher acoustic contrast, such as a higher mass density and/or a lower compressibility, thereby allowing the acoustic radiation pressure to push the first type of particles or biological entities 142 towards the pressure node located along the center of the main channel 102.

The fluid sample containing the first and second types of particles or biological entities 142 and 144 is introduced into the main channel 102 via the two side input channels 108 as two streams flowing near the sidewalls. The two streams of fluid sample in the main channel 102, which may behave like laminar flow, are interposed by the buffer fluid 146, which may act as a sheath fluid that may retard or prevent the movement of the second type of particles or biological entities 144 towards the pressure node along the center of the main channel 102. As the fluid sample progresses downstream in the main channel 102, the acoustic radiation pressure pushes the first type of particles or biological entities 142 towards the pressure node along the center of the main channel 102 while the second type of particles or biological entities 144 mostly remain close to the sidewalls. At the downstream end of the main channel 102, the first type of particles or biological entities 142 at the center exit the microfluidic device 100 through the center outlet port 110 and the second type of particles or biological entities 144 near the sidewalls are diverted to the side outlet port 112 through the side output channels 114.

The acoustic separation process illustrated in FIG. 3 may be sensitive to the power (e.g., voltage or current) applied to the piezoelectric transducers and the flow rate of the fluid sample containing the first and second types of particles or biological entities 142 and 144. Too high of power or too low of flow rate may cause some of the second type of particles or biological entities 144 to move into the center outlet port 110. The use of a buffer fluid with too low density and/or viscosity may also cause some of the second type of particles or biological entities 144 to move into the center outlet port 110. Conversely, too low of power or too high of flow rate may cause some of the first type of particles or biological entities 142 being diverted to the side outlet port 112 through the side output channels 114. The use of a buffer fluid with too high density and/or viscosity may also cause some of the first type of particles or biological entities 142 being diverted to the side outlet port 112.

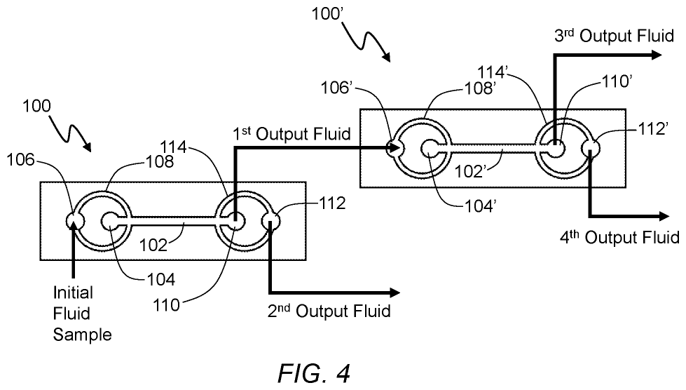

FIG. 4 shows the microfluidic device 100 connected to a first auxiliary microfluidic device 100′ in a serial configuration. In the drawing, numerals 102-114 denote the same components as those shown in FIGS. 2A and 3, and numerals 102′-114′ denote components analogous to those of 102-114; and the piezoelectric transducers are omitted for reasons of clarity. In the serial configuration comprising two microfluidic devices 100 and 100′ operating under the single pressure node condition, a fluid sample including therein first, second, and third types of particles or biological entities is introduced into the microfluidic device 100 through the side inlet port 106 for acoustic separation and a buffer fluid is introduced through the center inlet port 104. The first, second, and third types of particles or biological entities may have sufficiently different physical sizes and/or acoustic contrasts, allowing them to be separated into a first group comprising the first and second types of particles or biological entities and a second group comprising the third type of particles or biological entities when subjected to the acoustic radiation pressure in the main channel 102. For example, the first, second, and third types of particles or biological entities may have large, medium, and small physical sizes, respectively. In an embodiment, the average size or the median size of the third type of particles or biological entities is less than 1 μm. Alternatively, the first, second, and third types of particles or biological entities may differ in acoustic contrast, which depends on compressibility and density, thereby allowing them to be separated into two groups by the microfluidic device 100.

After entering the main channel 102 from the side input channels 108, the first group comprising the first and second types of particles or biological entities migrates towards the center of the main channel 102 from the sidewalls and exits the microfluidic device 100 through the center outlet port 110 as part of the first output fluid. The second group comprising the third type of particles or biological entities moves in two streams along the sidewalls of the main channel 102 and exits the microfluidic device 100 through the side outlet port 112 as part of the second output fluid. The first output fluid may have higher relative fractions of the first and second types of particles or biological entities than the initial fluid sample. The second output fluid may have lower relative fractions of the first and second types of particles or biological entities than the initial fluid sample.

After exiting the center outlet port 110 of the microfluidic device 100, the first output fluid is fed into the side inlet port 106′ of the first auxiliary microfluidic device 100′ for further acoustic separation of the first and second types of particles or biological entities, which have sufficiently different physical sizes and/or acoustic contrasts to allow them to be separated by the acoustic radiation pressure in the main channel 102′ under the single pressure node condition. For example, the first type of particles or biological entities may have a larger physical size and/or a higher acoustic contrast, such as a higher density and/or a lower compressibility, than the second type of particles or biological entities. A buffer fluid or a fluid not containing particles or biological entities is introduced through the center inlet port 104′ during the separation process.

After entering the main channel 102′ from the side input channels 108′, the first type of particles or biological entities in the first output fluid migrate towards the center of the main channel 102′ from the sidewalls under the acoustic radiation pressure and exit the first auxiliary microfluidic device 100′ through the center outlet port 110′ as part of the third output fluid. The second type of particles or biological entities in the first output fluid move in two streams along the sidewalls of the main channel 102′ and exit the first auxiliary microfluidic device 100′ through the side outlet port 112′ as part of the fourth output fluid. The third output fluid may have a higher relative fraction of the first type of particles or biological entities than the initial fluid sample and/or the first output fluid. The fourth output fluid may have a higher relative fraction of the second type of particles or biological entities than the initial fluid sample and/or the first output fluid.

In an embodiment, the microfluidic device 100 and the first auxiliary microfluidic device 100′ may be substantially identical but may have different operating conditions. For example, the buffer fluid used in the first auxiliary microfluidic device 100′ may have a higher viscosity and/or higher density than the buffer fluid used in the microfluidic device 100, or the power (e.g., voltage or current) applied to the piezoelectric transducers of the first auxiliary microfluidic device 100′ is lower than that applied to the piezoelectric transducers of the microfluidic device 100.

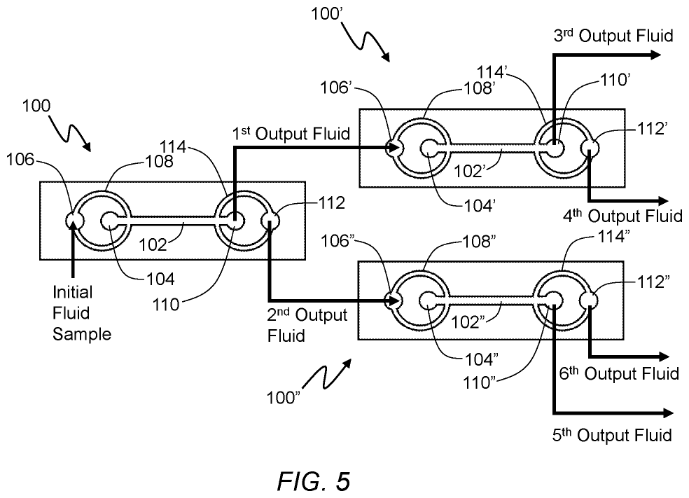

FIG. 5 shows that a second auxiliary microfluidic device 100″ may be added to the microfluidic device 100 and the first auxiliary microfluidic device 100′ to form a cascading configuration. The components 102″-114″ of the second auxiliary microfluidic device 100″ are analogous to the components 102-114 of the microfluidic device 100, respectively. In addition to being connected to the first auxiliary microfluidic device 100′ in series as shown in FIG. 4, the microfluidic device 100 may be connected to the second auxiliary microfluidic device 100″ in series. In the cascading configuration comprising three microfluidic devices 100, 100′, and 100″ operating under the single pressure node condition, a fluid sample including therein first, second, third, and fourth types of particles or biological entities is introduced into the microfluidic device 100 through the side inlet port 106 for acoustic separation and a buffer fluid is introduced through the center inlet port 104. The first, second, third, and fourth types of particles or biological entities may have sufficiently different physical sizes and/or acoustic contrasts, allowing them to be separated into a first group comprising the first and second types of particles or biological entities and a second group comprising the third and fourth types of particles or biological entities when subjected to the acoustic radiation pressure in the main channel 102. For example, the first, second, third, and fourth types of particles or biological entities may have large, medium, small, smallest physical sizes, respectively. Alternatively, the first, second, third, and fourth types of particles or biological entities may differ in acoustic contrast, which depends on compressibility and density, thereby allowing them to be separated into two groups by the microfluidic device 100.

After entering the main channel 102 from the side input channels 108, the first group comprising the first and second types of particles or biological entities migrates towards the center of the main channel 102 from the sidewalls and exits the microfluidic device 100 through the center outlet port 110 as part of the first output fluid. The second group comprising the third and fourth types of particles or biological entities moves in two streams along the sidewalls of the main channel 102 and exits the microfluidic device 100 through the side outlet port 112 as part of the second output fluid. The first output fluid may have higher relative fractions of the first and second types of particles or biological entities than the initial fluid sample. The second output fluid may have lower relative fractions of the first and second types of particles or biological entities than the initial fluid sample. The second output fluid may also have a lower relative fraction of the third type of particles or biological entities than the initial fluid sample.

After exiting the center outlet port 110 of the microfluidic device 100, the first output fluid is fed into the side inlet port 106′ of the first auxiliary microfluidic device 100′ for further acoustic separation of the first and second types of particles or biological entities, which have sufficiently different physical sizes and/or acoustic contrasts to allow them to be separated by the acoustic radiation pressure in the main channel 102′ under the single pressure node condition. For example, the first type of particles or biological entities may have a larger physical size and/or a higher acoustic contrast, such as a higher density and/or a lower compressibility, than the second type of particles or biological entities. A buffer fluid or a fluid not containing particles or biological entities is introduced through the center inlet port 104′ during the acoustic separation process.

After entering the main channel 102′ from the side input channels 108′, the first type of particles or biological entities in the first output fluid migrate towards the center of the main channel 102′ from the sidewalls under the acoustic radiation pressure and exit the first auxiliary microfluidic device 100′ through the center outlet port 110′ as part of the third output fluid. The second type of particles or biological entities in the first output fluid move in two streams along the sidewalls of the main channel 102′ and exit the first auxiliary microfluidic device 100′ through the side outlet port 112′ as part of the fourth output fluid. The third output fluid may have a higher relative fraction of the first type of particles or biological entities than the initial fluid sample and/or the first output fluid. The fourth output fluid may have a higher relative fraction of the second type of particles or biological entities than the initial fluid sample and/or the first output fluid.

After exiting the side outlet port 112 of the microfluidic device 100, the second output fluid is fed into the side inlet port 106″ of the second auxiliary microfluidic device 100″ for further acoustic separation of the third and fourth types of particles or biological entities, which have sufficiently different physical sizes and/or acoustic contrasts to allow them to be separated by the acoustic radiation pressure in the main channel 102″ under the single pressure node condition. For example, the third type of particles or biological entities may have a larger physical size and/or a higher acoustic contrast, such as a higher density and/or a lower compressibility, than the fourth type of particles or biological entities. A buffer fluid or a fluid not containing particles or biological entities is introduced through the center inlet port 104″ during the acoustic separation process.

After entering the main channel 102″ from the side input channels 108″, the third type of particles or biological entities in the second output fluid migrate towards the center of the main channel 102″ from the sidewalls under the acoustic radiation pressure and exit the second auxiliary microfluidic device 100″ through the center outlet port 110″ as part of the fifth output fluid. The fourth type of particles or biological entities in the second output fluid move in two streams along the sidewalls of the main channel 102″ and exit the second auxiliary microfluidic device 100″ through the side outlet port 112″ as part of the sixth output fluid. The fifth output fluid may have a higher relative fraction of the third type of particles or biological entities than the initial fluid sample and/or the second output fluid. The sixth output fluid may have a higher relative fraction of the fourth type of particles or biological entities than the initial fluid sample and/or the second output fluid.

In an embodiment, the microfluidic device 100 and the first and second auxiliary microfluidic devices 100′ and 100″ may be substantially identical but may have different operating conditions. For example and without limitation, the first auxiliary microfluidic devices 100′ may use a buffer fluid with a higher density and/or viscosity than the buffer fluid used in the microfluidic devices 100; and/or the second auxiliary microfluidic devices 100″ may use a buffer fluid with a lower density and/or viscosity than the buffer fluid used in the microfluidic devices 100; and/or the power (e.g., voltage or current) applied to the piezoelectric transducers of the first auxiliary microfluidic device 100′ is lower than that applied to the piezoelectric transducers of the microfluidic device 100; and/or the power (e.g., voltage or current) applied to the piezoelectric transducers of the second auxiliary microfluidic device 100″ is higher than that applied to the piezoelectric transducers of the microfluidic device 100.

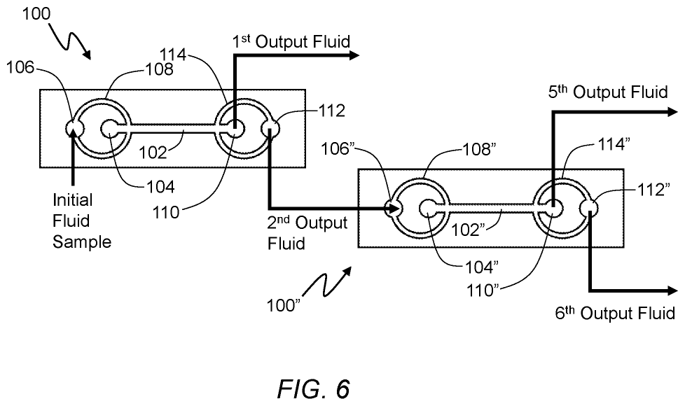

Referring now to FIG. 6, in another serial configuration comprising the microfluidic device 100 and the second auxiliary microfluidic device 100″ operating under the single pressure node condition, a fluid sample including therein the second, third, and fourth types of particles or biological entities is introduced into the microfluidic device 100 through the side inlet port 106 for acoustic separation and a buffer fluid is introduced through the center inlet port 104. The second, third, and fourth types of particles or biological entities may have sufficiently different physical sizes and/or acoustic contrasts, allowing them to be separated into a first group comprising the second type of particles or biological entities and a second group comprising the third and fourth types of particles or biological entities when subjected to the acoustic radiation pressure in the main channel 102. For example, the second, third, and fourth types of particles or biological entities may have large, medium, and small physical sizes, respectively. Alternatively, the second, third, and fourth types of particles or biological entities may differ in acoustic contrast, which depends on compressibility and density, thereby allowing them to be separated into two groups by the microfluidic device 100.

After entering the main channel 102 from the side input channels 108, the first group comprising the second type of particles or biological entities migrates towards the center of the main channel 102 from the sidewalls and exits the microfluidic device 100 through the center outlet port 110 as part of the first output fluid. The second group comprising the third and fourth types of particles or biological entities moves in two streams along the sidewalls of the main channel 102 and exits the microfluidic device 100 through the side outlet port 112 as part of the second output fluid. The first output fluid may have a higher relative fraction of the second type of particles or biological entities than the initial fluid sample. The second output fluid may have a lower relative fraction of the second type of particles or biological entities than the initial fluid sample. The second output fluid may also have a lower relative fraction of the third type of particles or biological entities than the initial fluid sample.

After exiting the side outlet port 112 of the microfluidic device 100, the second output fluid is fed into the side inlet port 106″ of the second auxiliary microfluidic device 100″ for further acoustic separation of the third and fourth types of particles or biological entities, which have sufficiently different physical sizes and/or acoustic contrasts to allow them to be separated by the acoustic radiation pressure in the main channel 102″ under the single pressure node condition. For example, the third type of particles or biological entities may have a larger physical size or a higher acoustic contrast, such as a higher density and/or a lower compressibility, than the fourth type of particles or biological entities. A buffer fluid or a fluid not containing particles or biological entities is introduced through the center inlet port 104″ during the acoustic separation process.

After entering the main channel 102″ from the side input channels 108″, the third type of particles or biological entities in the second output fluid migrate towards the center of the main channel 102″ from the sidewalls under the acoustic radiation pressure and exit the second auxiliary microfluidic device 100″ through the center outlet port 110″ as part of the fifth output fluid. The fourth type of particles or biological entities in the second output fluid move in two streams along the sidewalls of the main channel 102″ and exit the second auxiliary microfluidic device 100″ through the side outlet port 112″ as part of the sixth output fluid. The fifth output fluid may have a higher relative fraction of the third type of particles or biological entities than the initial fluid sample and/or the second output fluid. The sixth output fluid may have a higher relative fraction of the fourth type of particles or biological entities than the initial fluid sample and/or the second output fluid.

In an embodiment, the microfluidic device 100 and the second auxiliary microfluidic device 100″ may be substantially identical but may have different operating conditions. For example and without limitation, the second auxiliary microfluidic devices 100″ may use a buffer fluid with a lower density and/or viscosity than the buffer fluid used in the microfluidic devices 100; and/or the power (e.g., voltage or current) applied to the piezoelectric transducers of the second auxiliary microfluidic device 100″ is higher than that applied to the piezoelectric transducers of the microfluidic device 100.

In the serial and cascading configurations shown in FIGS. 4-6, the microfluidic device 100 and the first auxiliary microfluidic device 100′ may be fluidically coupled or connected by having a conduit connecting to the center outlet port 110 at one end and the side inlet port 106′ at the other end, thereby allowing the first output fluid to maintain the same flow rate between the two microfluidic devices 100 and 100′. Likewise, the microfluidic device 100 and the second auxiliary microfluidic device 100″ may be fluidically coupled or connected by having a conduit connecting to the center outlet port 110 at one end and the side inlet port 106″ at the other end, thereby allowing the second output fluid to maintain the same flow rate between the two microfluidic devices 100 and 100″.

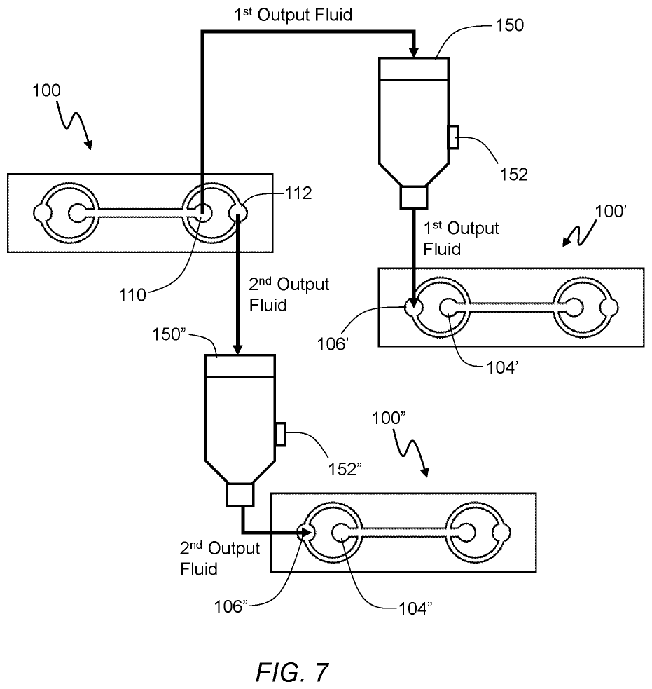

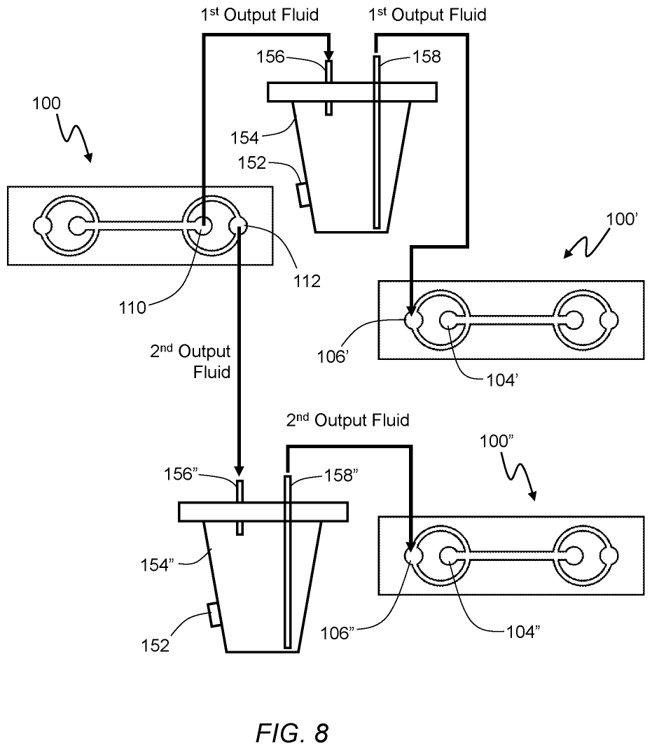

Alternatively, the microfluidic device 100 and the first auxiliary microfluidic device 100′ may be fluidically decoupled or disconnected by having a flow connector interposed between the center outlet port 110 and the side inlet port 106′ as shown in FIGS. 7-9, thereby allowing the fluid sample entering the side inlet port 106 of the microfluidic device 100 and the first output fluid entering the side inlet port 106′ of the first auxiliary microfluidic device 100′ to have independent flow rates for optimizing the acoustic separation processes in the respective devices 100 and 100′.

FIG. 7 shows a first type of flow connector 150 in the form of a liquid container interposed between the microfluidic device 100 and the first auxiliary microfluidic device 100′. A conduit may connect the center outlet port 110 of the microfluidic device 100 to the first type of flow connector 150 at the top thereof, and another conduit may connect the side inlet port 106′ of the first auxiliary microfluidic device 100′ to the first type of flow connector 150 at the bottom thereof. The first output fluid from the center outlet port 110 is fed into the first type of flow connector 150 at the top, and the first output fluid accumulated in the first type of flow connector 150 is drained from the bottom thereof into the side inlet port 106′ via gravity and/or with the assistance of a pump (not shown), thereby fluidically decoupling the incoming and outgoing fluids. The first type of flow connector 150 may further include a fluid level sensor 152 attached thereto or a remote sensor (not shown) for measuring the amount of the first output fluid accumulated in the first type of flow connector 150. The fluid level sensor 152 or the remote sensor may stop the flow of the first output fluid to the first auxiliary microfluidic device 100′ when the amount of the first output fluid in the first type of flow connector 150 drops below a critical level. The fluid level sensor 152 or the remote sensor may also stop the flow of the incoming first output fluid from the microfluidic device 100 when the amount of the first output fluid in the first type of flow connector 150 exceeds a critical level.

A first type of flow connector 150″ may also be interposed between the microfluidic device 100 and the second auxiliary microfluidic device 100″. A conduit may connect the side outlet port 112 of the microfluidic device 100 to the first type of flow connector 150″ at the top thereof, and another conduit may connect the side inlet port 106″ of the second auxiliary microfluidic device 100″ to the first type of flow connector 150″ at the bottom thereof. The second output fluid from the side outlet port 112 is fed into the first type of flow connector 150″ at the top, and the second output fluid accumulated in the first type of flow connector 150″ is drained from the bottom thereof into the side inlet port 106″ via gravity and/or with the assistance of a pump (not shown) , thereby fluidically decoupling the incoming and outgoing fluids. The first type of flow connector 150″ may further include a fluid level sensor 152″ attached thereto or a remote sensor for measuring the amount of the second output fluid accumulated in the first type of flow connector 150″.

FIG. 8 shows a second type of flow connector 154 in the form of a vial or container interposed between the microfluidic device 100 and the first auxiliary microfluidic device 100′. A conduit may connect the center outlet port 110 of the microfluidic device 100 to an inlet tube 156 at the top of the second type of flow connector 154, and another conduit may connect the side inlet port 106′ of the first auxiliary microfluidic device 100′ to an outlet tube 158 at the top of the second type of flow connector 154. The inlet tube 156 may have a relatively short length and may not touch the first output fluid accumulated in the second type of flow connector 154, thereby allowing the incoming first output fluid to drip into the second type of flow connector 154 from the top thereof via gravity. The outlet tube 158 may have a sufficient length spanning from the top of the second type of flow connector 154 to near the bottom thereof, thereby allowing the downstream processing by the first auxiliary microfluidic device 100′ with a small amount of the first output fluid accumulated in the second type of flow connector 154. The second type of flow connector 154 allows the incoming first output fluid and the outgoing first output fluid to be fluidically decoupled. During operation of the first auxiliary microfluidic device 100′, the first output fluid accumulated in the second type of flow connector 154 is discharged through the top thereof into the side inlet port 106′ via gravity and/or with the assistance of a pump (not shown). The second type of flow connector 154 may further include a fluid level sensor 152 attached thereto or a remote sensor (not shown) for measuring the amount of the first output fluid accumulated in the second type of flow connector 154. The fluid level sensor 152 or the remote sensor may stop the flow of the first output fluid to the first auxiliary microfluidic device 100′ when the amount of the first output fluid in the second type of flow connector 154 drops below a critical level. The fluid level sensor 152 or the remote sensor may also stop the flow of the incoming first output fluid from the microfluidic device 100 when the amount of the first output fluid in the second type of flow connector 154 exceeds a critical level.

A second type of flow connector 154″ may also be interposed between the microfluidic device 100 and the second auxiliary microfluidic device 100″. A conduit may connect the side outlet port 112 of the microfluidic device 100 to an inlet tube 156″ at the top of the second type of flow connector 154″, and another conduit may connect the side inlet port 106″ of the second auxiliary microfluidic device 100″ to an outlet tube 158″ at the top of the second type of flow connector 154″. The inlet tube 156″ may have a relatively short length and may not touch the second output fluid accumulated in the second type of flow connector 154″, thereby allowing the incoming first output fluid to drip into the second type of flow connector 154″ from the top thereof via gravity. The outlet tube 158″ may have a sufficient length spanning from the top of the second type of flow connector 154″ to near the bottom thereof, thereby allowing the downstream processing by the second auxiliary microfluidic device 100″ with a small amount of the second output fluid accumulated in the second type of flow connector 154″. The second type of flow connector 154″ allows the incoming second output fluid and the outgoing second output fluid to be fluidically decoupled. During operation of the second auxiliary microfluidic device 100″, the second output fluid accumulated in the second type of flow connector 154″ is discharged through the top thereof into the side inlet port 106″ via gravity and/or with the assistance of a pump (not shown). The second type of flow connector 154″ may further include a fluid level sensor 152″ attached thereto or a remote sensor (not shown) for measuring the amount of the second output fluid accumulated in the second type of flow connector 154″.

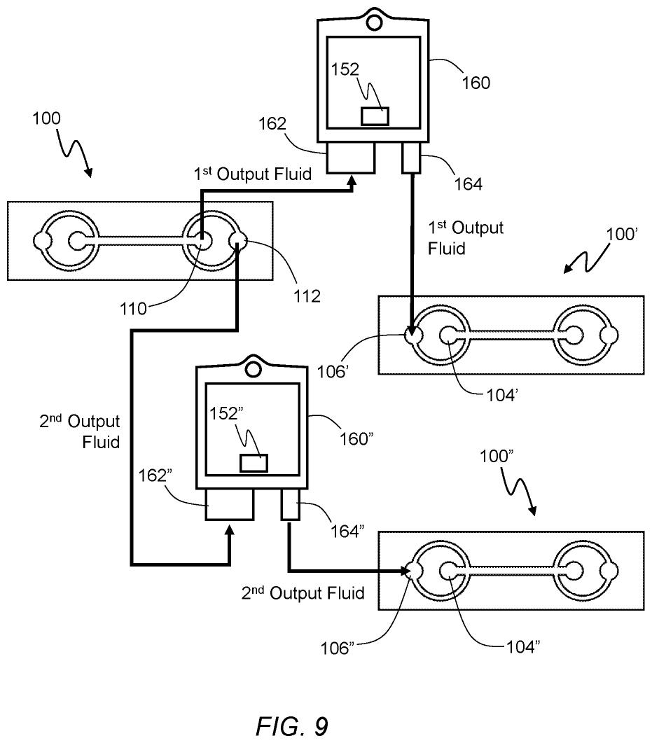

FIG. 9 shows a third type of flow connector 160 in the form of a pliable fluid bag or pliable blood bag interposed between the microfluidic device 100 and the first auxiliary microfluidic device 100′. A conduit may connect the center outlet port 110 of the microfluidic device 100 to the third type of flow connector 160 through a bottom inlet 162 thereof, and another conduit may connect the side inlet port 106′ of the first auxiliary microfluidic device 100′ to the third type of flow connector 160 through a bottom outlet 164 thereof. The first output fluid from the center outlet port 110 is fed into the third type of flow connector 160 at the bottom, and the first output fluid accumulated in the third type of flow connector 160 is drained from the bottom thereof into the side inlet port 106′ via gravity and/or with the assistance of a pump (not shown). The third type of flow connector 160 may further include a fluid level sensor 152 attached thereto or a remote sensor (not shown) for measuring the amount of the first output fluid accumulated in the third type of flow connector 160. The fluid level sensor 152 or the remote sensor may stop the flow of the first output fluid to the first auxiliary microfluidic device 100′ when the amount of the first output fluid in the third type of flow connector 160 drops below a critical level. The fluid level sensor 152 or the remote sensor may also stop the flow of the incoming first output fluid from the microfluidic device 100 when the amount of the first output fluid in the third type of flow connector 160 exceeds a critical level.

A third type of flow connector 160″ may also be interposed between the microfluidic device 100 and the second auxiliary microfluidic device 100″. A conduit may connect the side outlet port 112 of the microfluidic device 100 to the third type of flow connector 160″ through a bottom inlet 162″ thereof, and another conduit may connect the side inlet port 106″ of the second auxiliary microfluidic device 100″ to the third type of flow connector 160″ through a bottom outlet 164″ thereof. The second output fluid from the side outlet port 112 is fed into the third type of flow connector 160″ through the bottom thereof, and the second output fluid accumulated in the third type of flow connector 160″ is drained from the bottom thereof into the side inlet port 106″ via gravity and/or with the assistance of a pump (not shown). The third type of flow connector 160″ may further include a fluid level sensor 152″ attached thereto or a remote sensor (not shown) for measuring the amount of the first output fluid accumulated in the third type of flow connector 160″.

The first, second, and third types of flow connectors 150, 154, and 160 may serve as reservoirs for accumulating the first output fluid from the microfluidic device 100. The first auxiliary microfluidic device 100′ may operate after all of the initial fluid sample is processed through the microfluidic device 100 or after a certain amount of the first output fluid is accumulated in the flow connectors 150, 154, and 160. The use of the flow connectors 150, 154, and 160 between the microfluidic device 100 and the first auxiliary microfluidic device 100′ allows the two microfluidic devices 100 and 100′ to independently operate at their respective optimal flow rates, which may be different. In addition to the possibilities of using buffer fluids with different physical properties and different powers for the piezoelectric transducers as described above, the microfluidic devices 100 and 100′ with one of the exemplary flow connectors 150, 154, and 160 interposed therebetween may operate with independent flow rates. In an embodiment, the first output fluid is fed into the side inlet port 106′ of the first auxiliary microfluidic device 100′ at a higher flow rate than the initial fluid sample being fed into the side inlet port 106 of the microfluidic device 100.

Likewise, the first, second, and third types of flow connectors 150″, 154″, and 160″ may serve as reservoirs for accumulating the second output fluid from the microfluidic device 100. The second auxiliary microfluidic device 100″ may operate after all of the initial fluid sample is processed through the microfluidic device 100 or after a certain amount of the second output fluid is accumulated in the flow connectors 150″, 154″, and 160″. The use of the flow connectors 150″, 154″, and 160″ between the microfluidic device 100 and the second auxiliary microfluidic device 100″ allows the two microfluidic devices 100 and 100″ to independently operate at their respective optimal flow rates, which may be different. In addition to the possibilities of using buffer fluids with different physical properties and different powers for the piezoelectric transducers as described above, the microfluidic devices 100 and 100″ with one of the exemplary flow connectors 150″, 154″, and 160″ interposed therebetween may operate with independent flow rates. In an embodiment, the second output fluid is fed into the side inlet port 106″ of the second auxiliary microfluidic device 100″ at a lower flow rate than the initial fluid sample being fed into the side inlet port 106 of the microfluidic device 100.

Any one of the flow connectors 150, 150″, 154, 154″, 160, and 160″ and the conduits connected thereto may be assembled or integrated as a unit and packaged in a sterile environment or sterilized after packaging (e.g., UV light sterilization), thereby minimizing potential contamination when operating the microfluidic devices 100, 100′, and 100″ in a clinical environment.

In the serial and cascading configurations shown in FIGS. 4 and 5, the microfluidic device 100 and the first auxiliary microfluidic device 100′ may be fluidically decoupled by having one of the flow connectors 150, 154, and 160 interposed between the microfluidic devices 100 and 100′, thereby allowing the first output fluid exiting the center outlet port 110 of the microfluidic device 100 and the first output fluid entering the side inlet port 106′ of the first auxiliary microfluidic device 100′ to have independent flow rates. In an embodiment, the first output fluid exiting the center outlet port 110 has a lower flow rate than the first output fluid entering the side inlet port 106′.

In the cascading and serial configurations shown in FIGS. 5 and 6, the microfluidic device 100 and the second auxiliary microfluidic device 100″ may be fluidically decoupled by having one of the flow connectors 150″, 154″, and 160″ interposed between the microfluidic devices 100 and 100″, thereby allowing the second output fluid exiting the side outlet port 112 of the microfluidic device 100 and the second output fluid entering the side inlet port 106″ of the second auxiliary microfluidic device 100″ to have independent flow rates. In an embodiment, the second output fluid exiting the side outlet port 112 has a higher flow rate than the second output fluid entering the side inlet port 106″.

In the cascading configuration shown in FIG. 5, the microfluidic device 100 and the first auxiliary microfluidic device 100′ may be fluidically coupled via a conduit or fluidically decoupled by utilizing any one of the flow connectors 150, 154, and 160; and the microfluidic device 100 and the second auxiliary microfluidic device 100″ may independently be fluidically coupled via a conduit or fluidically decoupled by utilizing any one of the flow connectors 150″, 154″, and 160″.

It is worth noting that the present invention may be practiced with alternative flow connector designs that can serve as reservoirs to accumulate the first or second output fluid and/or fluidically decouple the downstream microfluidic device from the upstream microfluidic device by interrupting the fluid path therebetween, thereby allowing the upstream and downstream microfluidic devices to operate with independent flow rates.

Embodiments of the present invention that utilize the serial and cascading configurations illustrated in FIGS. 4-6 may be used to enrich or extract a type or group of biological entities among multiple types or groups of biological entities from an initial fluid sample. For example and without limitation, the serial configuration shown in FIG. 6 may be used to enrich or extract tumor infiltrating lymphocyte (TIL) cells or peripheral blood mononuclear cells (PBMCs) from an initial sample.

### EXAMPLE 1

Tumor Infiltrating Lymphocytes (TILs), which may eradicate tumor cells and are being investigated as a cellular immunotherapy, may often be found in the tumor stroma and within the tumor itself. Therefore, TILs may be extracted from a sample containing multiple types of cells from dissociated tumor tissue.

An initial fluid sample containing TILs for the acoustic particle separation process was prepared using a tissue sample from a lung tumor biopsy. The tissue sample was cut by a pair of aseptic scissors and minced with aseptic blades on an aseptic surface. The minced tissue sample was dissociated into individual cells by incubation in a dissociation buffer manufactured by Singleron Biotechnologies for 45 min at 37° C. The cells in the dissociation buffer were then stained with CD45 AF488 and EpCAM PE fluorescent antibodies and incubated for 20 min at room temperature in a dark environment. A lysing buffer manufactured by Solarbio for lysing red blood cells (RBCs) was added to the dissociation buffer with the cells therein. The entire mixture was incubated for 15 min at room temperature in a dark environment and then strained using a 40 μm cell strainer to remove large tissue aggregates that were not properly dissociated, thereby yielding the initial fluid sample for acoustic separation comprising TILs, tumor cells, and organic debris, such as cell membranes and dead cells, suspended in a mixture of the dissociation and lysing buffers.

The tumor cells labeled by the EpCAM PE antibody, the TILs labeled by the CD45 AF488 antibody, and the organic debris not labeled in the initial fluid sample were quantified using a flow cytometer (CytoFlex, Beckman Coulter).

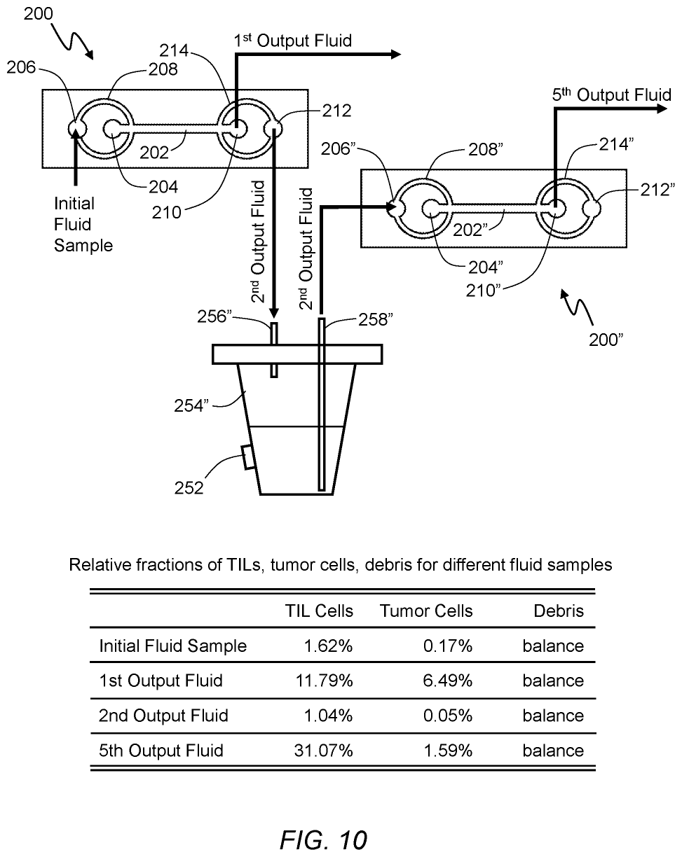

The separation process begins by introducing the initial fluid sample, which contains TILs that accounted for 1.62% relative fraction of all biological entities detected by the cytometer, tumor cells that accounted for 0.17% relative fraction of all biological entities detected by the cytometer, and debris that accounted for the balance of all biological entities detected by the cytometer, into an upstream microfluidic device 200 through a side inlet port 206 at a flow rate of 1.0 ml/min with a buffer fluid (MARS Wash Buffer, Applied Cells Inc.) being introduced through a center inlet port 204 at a flow rate of 1.0 ml/min as shown in FIG. 10. A peak-to-peak voltage of 14 V with a frequency of 2 MHz is applied to the piezoelectric transducers of the microfluidic device 200 to generate a single pressure node in the main channel 202. The first output fluid, which contains TILs at 11.79% relative fraction and tumor cells at 6.49% relative fraction of all biological entities detected by the cytometer, is extracted from a center outlet port 210. The second output fluid, which contains TILs at 1.04% relative fraction and tumor cells at 0.05% relative fraction, is extracted from a side outlet port 212.

The extracted second output fluid is fed into a second type of flow connector 254″, which allows the second output fluid exiting the upstream microfluidic device 200 and the second output fluid entering a downstream microfluidic device 200″ to have independent flow rates. The second output fluid accumulated in the second type of flow connector 254″ is then fed into the downstream microfluidic device 200″ through a side inlet port 206″ at a flow rate of 1.0 ml/min with the buffer fluid (MARS Wash Buffer, Applied Cells Inc.) being introduced through a center inlet port 204″ at a flow rate of 1.0 ml/min.

The downstream microfluidic device 200″ is substantially identical to the upstream microfluidic device 200 but uses a different operating voltage—a higher peak-to-peak voltage of 19 V with a frequency of 2 MHz is applied to the piezoelectric transducers to generate a single pressure node in the main channel 202″. The fifth output fluid exiting a center outlet port 210″ of the downstream microfluidic device 200″ contains TILs at 31.07% relative fraction, which is much higher than the initial relative fraction of 1.62% and is about 3 times that obtained through the conventional enrichment method of repeated centrifugation (3×).

### EXAMPLE 2

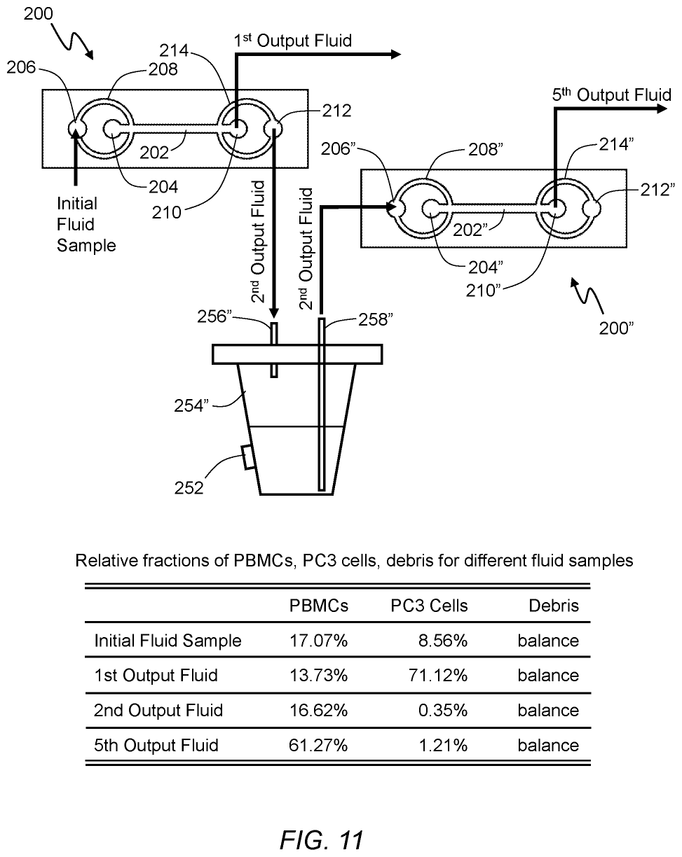

The same serial configuration shown in FIG. 10 is used to enrich the peripheral blood mononuclear cells (PBMCs) in an initial fluid sample containing PBMCs that accounted for 17.07% relative fraction of all biological entities detected by the cytometer, PC3 cancer cells that accounted for 8.56% relative fraction of all biological entities detected by the cytometer, and debris that accounted for the balance of all biological entities detected by the cytometer. The operating conditions of the upstream and downstream microfluidic devices 200 and 200″ are substantially similar to those of Example 1 described above. The initial fluid sample containing PBMCs and PC3 cancer cells is introduced into the upstream microfluidic device 200 through a side inlet port 206 at a flow rate of 1.0 ml/min with the buffer fluid being introduced through the center inlet port 204 at a flow rate of 1.0 ml/min as shown in FIG. 11. A peak-to-peak voltage of 14 V with a frequency of 2 MHz is applied to the piezoelectric transducers of the microfluidic device 200 to generate a single pressure node in the main channel 202. The first output fluid, which contains PBMCs at 13.73% relative fraction and PC3 cancer cells at 71.12% relative fraction, is extracted from the center outlet port 210. The second output fluid, which contains PBMCs at 16.62% relative fraction and PC3 cancer cells at 0.35% relative fraction, is extracted from the side outlet port 212.

The extracted second output fluid is fed into the second type of flow connector 254″, which allows the second output fluid exiting the upstream microfluidic device 200 and the second output fluid entering the downstream microfluidic device 200″ to have independent flow rates. The second output fluid accumulated in the second type of flow connector 254″ is then fed into the downstream microfluidic device 200″ through a side inlet port 206″ at a flow rate of 1.0 ml/min with the buffer fluid being introduced through the center inlet port 204″ at a flow rate of 1.0 ml/min.

The downstream microfluidic device 200″ is substantially identical to the upstream microfluidic device 200 but uses a different operating voltage—a higher peak-to-peak voltage of 19 V with a frequency of 2 MHz is applied to the piezoelectric transducers to generate a single pressure node in the main channel 202″. The fifth output fluid exiting the center outlet port 210″ of the downstream microfluidic device 200″ contains PBMCs at 61.27% relative fraction, which is higher than the initial relative fraction of 17.07%, while minimizing the relative fraction of PC3 cancer cells to 1.21%.

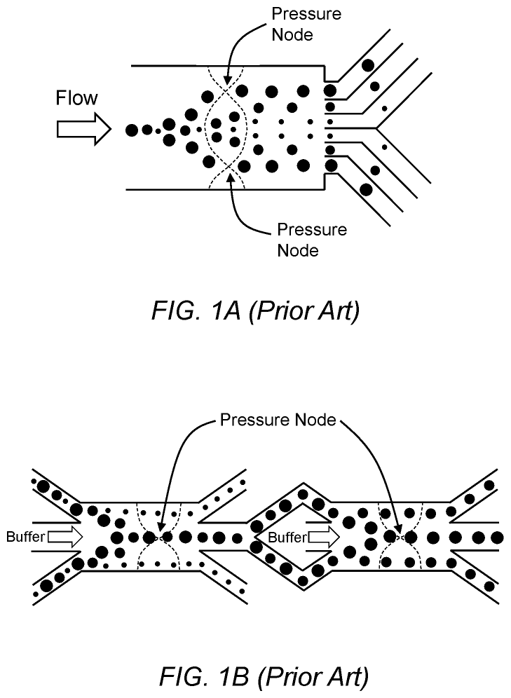

The operating conditions for the upstream and downstream microfluidic devices 200 and 200″ as described above are examples only and do not limit the scope of the present invention. It is worth noting that in the above examples, the upstream and downstream microfluidic devices 200 and 200″ independently operate at a sample flow rate of 60 ml/hr, which is 30 times faster than the sample flow rate of 2 ml/hr used in the two-stage microfluidic device of Adams et al. shown in FIG. 1B.

In addition to the examples described above, the present invention may be used to separate other fluid samples containing multiple types or groups of biological entities. For example, the cascading configuration shown in FIGS. 5 and 7-9 may be used to separate a fluid sample containing dissociated tumor tissue, which includes tumor cell clusters, individual tumor cells, fibroblast cells, and infiltrating immune cells. The initial fluid sample containing tumor cell clusters, individual tumor cells, fibroblast cells, and infiltrating immune cells may be introduced into the microfluidic device 100 through the side inlet port 106 with a first buffer fluid being introduced through the center inlet port 104. The first output fluid containing the tumor cell clusters and individual tumor cells is extracted from the center outlet port 110 and introduced into the first auxiliary microfluidic device 100′ through the side inlet port 106′, with a second buffer fluid being introduced through the center inlet port 104′. The second output fluid containing the fibroblast cells and infiltrating immune cells is extracted from the side outlet port 112 and introduced into the second auxiliary microfluidic device 100″ through the side inlet port 106″, with a third buffer fluid being introduced through the center inlet port 104″. The third output fluid containing the tumor cell clusters is extracted from the center outlet port 110′. The fourth output fluid containing the individual tumor cells is extracted from the side outlet port 112′. The fifth output fluid containing the fibroblast cells is extracted from the center outlet port 110″. The sixth output fluid containing the infiltrating immune cells is extracted from the side outlet port 112″.

While the present invention has been shown and described with reference to certain preferred embodiments, it is to be understood that those skilled in the art will no doubt devise certain alterations and modifications thereto which nevertheless include the true spirit and scope of the present invention. Thus the scope of the invention should be determined by the appended claims and their legal equivalents, rather than by examples given.

Any element in a claim that does not explicitly state “means for” performing a specified function, or “step for” performing a specific function, is not to be interpreted as a “means” or “step” clause as specified in 35 U.S.C. § 112, ¶ 6. In particular, the use of “step of” in the claims herein is not intended to invoke the provisions of 35 U.S.C. § 112, ¶ 6.

What is claimed is:

1. A method for separating biological entities in a fluid comprising the steps of:
introducing an initial fluid sample into a first microfluidic device as two streams along two sidewalls of a first linear channel at an upstream end thereof with a first buffer fluid interposed between the two streams of the initial fluid sample, the initial fluid sample including tumor cells and tumor infiltrating lymphocyte (TIL) cells;
applying a first power to the first microfluidic device to exert a first acoustic radiation pressure on the initial fluid sample and the first buffer fluid flowing in the first linear channel to produce a first output fluid sample exiting the first microfluidic device along a center of the first linear channel and a second output fluid sample exiting the first microfluidic device along the two sidewalls of the first linear channel, the first output fluid sample having a higher relative fraction of the tumor cells than the initial fluid sample and the second output fluid sample having a lower relative fraction of the tumor cells than the initial fluid sample;
flowing the second output fluid sample from the first microfluidic device into a flow connector;
flowing the second output fluid sample accumulated in the flow connector into a second microfluidic device as two streams along two sidewalls of a second linear channel at an upstream end thereof with a second buffer fluid interposed between the two streams of the second output fluid sample; and
applying a second power to the second microfluidic device to exert a second acoustic radiation pressure on the second output fluid sample and the second buffer fluid flowing in the second linear channel to produce a third output fluid sample exiting the second microfluidic device along a center of the second linear channel, the third output fluid having a higher relative fraction of TIL cells than the initial fluid sample,
wherein the second power is higher than the first power, and
wherein a flow rate of the second output fluid sample exiting the first microfluidic device is decoupled from a flow rate of the second output fluid sample entering the second microfluidic device.

2. The method of claim 1, wherein the second output fluid sample has a lower relative fraction of the TIL cells than the initial fluid sample.

3. The method of claim 1, wherein the first microfluidic device includes a substrate with the first linear channel formed therein, a lid on top of the substrate that covers the first linear channel, and one or more piezoelectric transducers attached to a surface of the lid opposite the substrate.

4. The method of claim 1, wherein the second microfluidic device includes a substrate with the second linear channel formed therein, a lid on top of the substrate that covers the second linear channel, and one or more piezoelectric transducers attached to a surface of the lid opposite the substrate.

5. The method of claim 1, wherein the first and second microfluidic devices are substantially identical.

6. The method of claim 1, wherein the first and second acoustic radiation pressures are respectively generated by acoustic standing waves having single pressure node.

7. A method for separating biological entities in a fluid comprising the steps of:
introducing an initial fluid sample into a first microfluidic device as two streams along two sidewalls of a first linear channel at an upstream end thereof with a first buffer fluid interposed between the two streams of the initial fluid sample, the initial fluid sample including cancer cells and peripheral blood mononuclear cells (PBMCs);
applying a first power to the first microfluidic device to exert a first acoustic radiation pressure on the initial fluid sample and the first buffer fluid flowing in the first linear channel to produce a first output fluid sample exiting the first microfluidic device along a center of the first linear channel and a second output fluid sample exiting the first microfluidic device along the two sidewalls of the first linear channel, the first output fluid sample having a higher relative fraction of the cancer cells than the initial fluid sample and the second output fluid sample having a lower relative fraction of the cancer cells than the initial fluid sample;
flowing the second output fluid sample from the first microfluidic device into a flow connector;
flowing the second output fluid sample accumulated in the flow connector into a second microfluidic device as two streams along two sidewalls of a second linear channel at an upstream end thereof with a second buffer fluid interposed between the two streams of the second output fluid sample; and
applying a second power to the second microfluidic device to exert a second acoustic radiation pressure on the second output fluid sample and the second buffer fluid flowing in the second linear channel to produce a third output fluid sample exiting the second microfluidic device along a center of the second linear channel, the third output fluid having a higher relative fraction of PBMCs than the initial fluid sample,
wherein the second power is higher than the first power, and
wherein a flow rate of the second output fluid sample exiting the first microfluidic device is decoupled from a flow rate of the second output fluid sample entering the second microfluidic device.

8. The method of claim 7, wherein the second output fluid sample has a lower relative fraction of the PBMCs than the initial fluid sample.

9. The method of claim 7, wherein the first microfluidic device includes a substrate with the first linear channel formed therein, a lid on top of the substrate that covers the first linear channel, and one or more piezoelectric transducers attached to a surface of the lid opposite the substrate.

10. The method of claim 7, wherein the second microfluidic device includes a substrate with the second linear channel formed therein, a lid on top of the substrate that covers the second linear channel, and one or more piezoelectric transducers attached to a surface of the lid opposite the substrate.

11. The method of claim 7, wherein the first and second microfluidic devices are substantially identical.

12. The method of claim 7, wherein the first and second acoustic radiation pressures are respectively generated by acoustic standing waves having single pressure node.
